(19) United States
(12) Patent Application Publication
(10) Pub. No.: US 2026/0363941 A1
(43) Pub. Date: Nov. 19, 2026

(54) THERAPEUTIC COMPRESSION DEVICE COMPRISING A DEVICE FOR ADJUSTMENT OF THE EXERTED PRESSURE

(71) Applicant: THUASNE, LEVALLOIS PERRET (FR)

(72) Inventors: Florence ROLLET, SAINT GENEST-LERPT (FR); Angélique FAVIER, SAINT JUST SAINT RAMBERT (FR)

(21) Appl. No.: 19/136,787

(22) PCT Filed: Dec. 11, 2023
(86) PCT No.: PCT/EP2023/085099

§ 371 Date: Jun. 6, 2025

(30) Foreign Application Priority Data
Dec. 12, 2022 (FR) FR2213167

(51) Int. Cl. A61H 11/00

(52) U.S. Cl. CPC A61H 11/00; A61H 2201/0192; A61H 2209/00

(57) ABSTRACT

The compression device (1) comprises at least one body (2), a free end (7) of an extensible part (4) of the body (2) being attachable in a plurality of attachment positions on the body (2), said compression device further comprising a pressure adjustment device (14).

The adjustment device (14) comprises an inextensible tab (16) attached on the body (2) and a loop (18) extending on or in the body (2), said tab (16) being engaged in said loop (18) and moving with respect to the loop (18) in the direction of extensibility of the extensible part (4) depending on the position of attachment of the free end (7) of the extensible part (4), the position of the tab (16) with respect to the loop (18) indicating the pressure level exerted by the compression device.

The present invention relates to a therapeutic compression device for a part of a patient's limb, of the type comprising at least one body at least partially extensible, a free end of an extensible part of the body being attachable in a plurality of attachment positions on the body around the limb part, the pressure exerted by the compression device varying according to the position of the free end of the extensible part on the body, said compression device further comprising at least one device for adjusting the pressure exerted by the compression device indicating at least one level of pressure exerted by the compression device according to the attachment position of the free end of the extensible part.

Such a compression device applied to a limb of a patient allows, in particular, to combat vascular overpressure and liquid stasis when the patient suffers from a vascular pathology, such as chronic venous disease, venous thrombosis, lymphedema, etc. Such a device can also be used in traumatology or orthopedics to maintain the limb in certain positions.

The device comprises, e.g., several extensible textile bands wrapped around a part of the limb to be treated and closed on themselves or on a body from which they extend. The tightening of the bands around the limb part allows pressure to be applied on said limb, said pressure being variable depending on the degree of tightening of the bands. For the treatment to be effective, the pressure exerted must be adjusted relatively precisely, applied for long periods of time, and repeatedly. The compression device must be placed to allow such pressure to be applied. Thus, the compression device must allow the pressure exerted by the different bands to be known according to their tightening to allow this pressure to be adjusted, and this adjustment must be repeatable when the compression device is removed and then put back on the limb part.

The compression device comprises, e.g., indices allowing the level of pressure applied to be indicated by aligning an index carried by one end of a band with an index placed in the attachment area of this band end on the band itself or on a body from which the band extends. These indices must allow the person applying the compression device, generally the patient themselves or a healthcare professional, to apply the desired pressure reproducibly.

However, such indices do not give complete satisfaction, particularly for reasons of readability. Indeed, the indices are not necessarily placed in a very visible location for the person applying the compression device, especially when the patient themselves performs the operation. This difficulty is further accentuated when the compression device is applied to a part of the patient's arm, with the patient having difficulty seeing the indices and aligning the indices appropriately.

One of the aims of the invention is to overcome this drawback by proposing a compression device comprising a pressure adjustment device that is easy to use and the readability of which is improved.

To this end, the invention relates to a compression device of the aforementioned type, wherein said adjustment device comprises at least one inextensible tab attached on the body and at least one loop extending on or in the body, at least one of the tab and the loop extending in the extensible part, said tab being engaged in said loop and moving relative to the loop along the direction of extensibility of the extensible part depending on the attachment position of the free end of the extensible part, the position of the tab relative to the loop indicating the level of pressure exerted by the compression device corresponding to the attachment position of the free end of the extensible part on the body.

The tab and the loop provide particularly visible and accessible information on the pressure exerted by the compression device, allowing for precise and reproducible adjustment during the tightening of the compression device. Indeed, the loop forms a marker as visible as the marker is tactile, allowing the person applying the compression device to easily adjust the pressure, even if this person is the patient themselves.

The compression device according to the invention can further comprise one or a plurality of the following features, taken individually or in any technically conceivable combination:
the tab and the loop extend in the extensible part of the body;
the tab comprises at least one index, the position of the index relative to the loop indicating the level of pressure exerted by the compression device corresponding to the attachment position of the free end of the extensible part on the body;
the tab comprises at least two indices, spaced apart along the direction of extensibility of the extensible part, the position of each index corresponding to a predetermined level of pressure exerted by the compression device when said index is aligned with the loop;
the tab extends between a first end attached on the body and a second end not attached on the body, the tab extending in the loop between the first end thereof and the second end thereof;
the second end of the tab comprises a stop element preventing the extraction of the tab from the loop;
the body is formed by an extensible band that can be wrapped around the limb part and attached thereto in a plurality of attachment positions around the limb part;
the compression device comprises several bodies each formed by an extensible band, said bodies being attached on each other and each being able to be wrapped around a different limb part, each body comprising a pressure adjustment device;
the body comprises an inextensible part and at least one extensible part, the extensible part protruding from an edge of said inextensible part and being attachable in a plurality of attachment positions onto the inextensible part;
the body comprises a plurality of extensible parts protruding from at least one edge of the inextensible part, each extensible part being attachable in a plurality of attachment positions onto the inextensible part, a pressure adjustment device being provided for each extensible part.

Other aspects and advantages of the invention will appear upon reading the following description, given by way of example and with reference to the appended drawings, wherein:

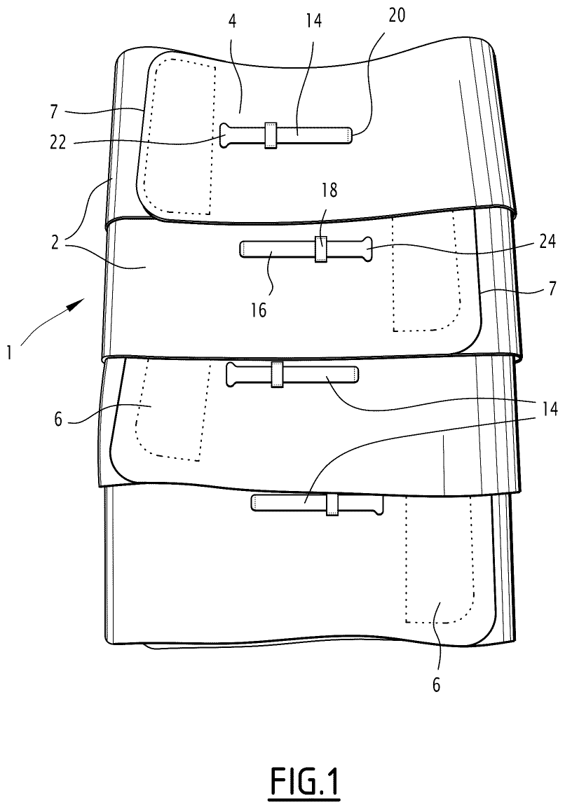

FIG. 1 is a schematic front view representation of a compression device according to one embodiment of the invention, in a closed position,

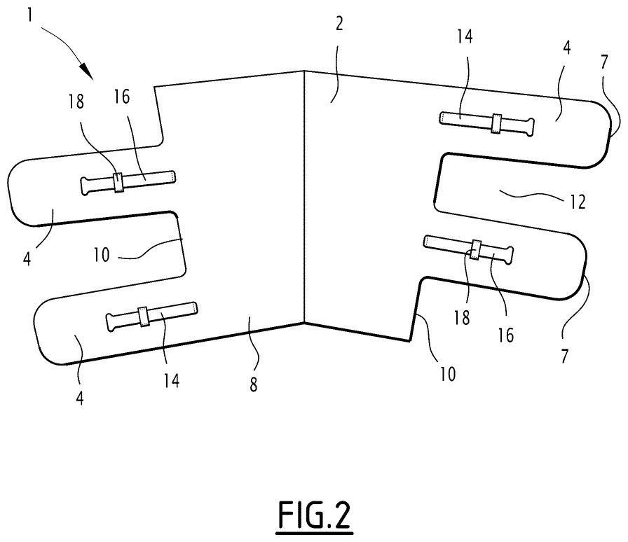

FIG. 2 is a schematic rear-view representation of a compression device according to another embodiment of the invention in an open position,

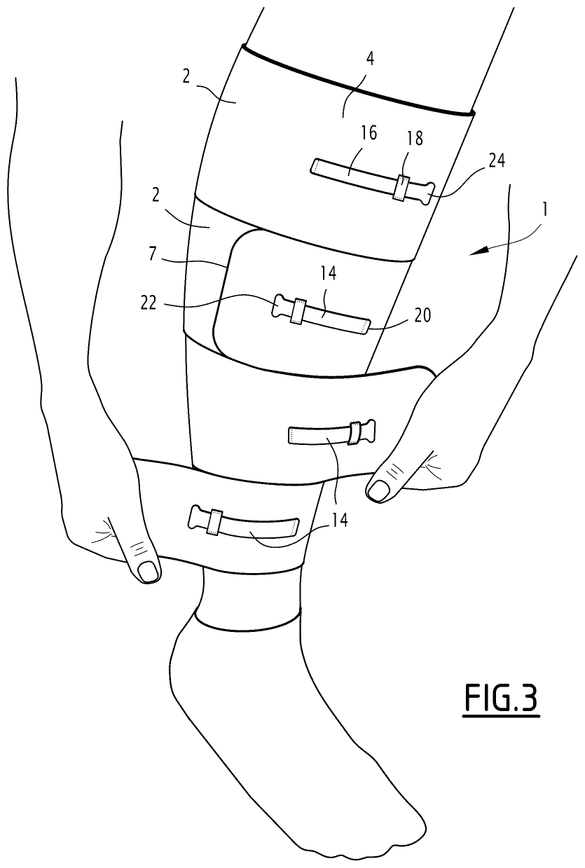

FIG. 3 is a schematic perspective representation of the compression device of FIG. 2 during the application thereof on a limb part,

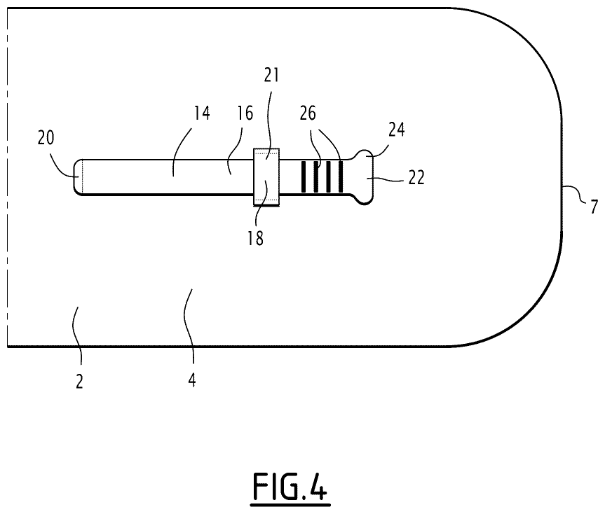

FIG. 4 is a schematic perspective representation of a pressure adjustment device according to one embodiment for a compression device according to the invention, and

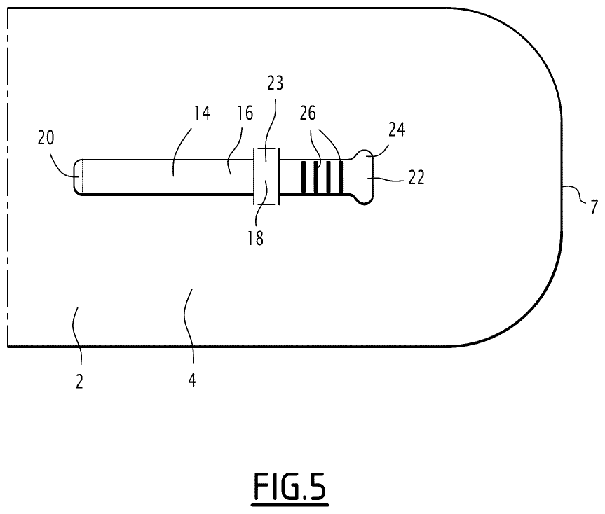

FIG. 5 is a schematic perspective representation of a pressure adjustment device according to another embodiment for a compression device according to the invention.

Referring to FIGS. 1 and 2, a compression device 1 is described comprising at least one body 2 at least partially extensible. At least partially extensible, means that the body 2 comprises at least one extensible part 4 that can be stretched in a direction of extensibility. Extensible, means a part that can be stretched to increase the dimension thereof along the direction of extensibility. According to one embodiment, the extensible part is also elastic so that said part substantially returns to the initial shape thereof when the force tending to stretch said part ceases to be applied. Extensibility can be obtained due to the material(s) used to make the extensible part, e.g., an elastic material, and/or due to a treatment of the extensible part, e.g., crimping or other. Extensibility corresponds, e.g., to an elongation comprised between 30% and 50%, e.g., substantially equal to 40% of the dimension of the extensible part along the direction of extensibility. The body 2 is, e.g., made of textile material, e.g., a textile complex comprising two layers of woven or knitted textile and a layer of open-cell foam padding or neoprene extending between the two layers of woven or knitted textile.

The extensible part 4 extends, e.g., to at least one extreme part of the body 2 along the direction of extensibility.

The compression device further comprises at least one attachment device 6 arranged to allow the attachment of a free end 7 of the extensible part 4 onto another part of the body 2. Free end 7, means the end of the extensible part 4 that is not attached on the rest of the body 2. The attachment device 6 is more particularly arranged so that a part of the body 2 comprising the extensible part 4 having made at least one turn around a limb part can be attached on another part of the body 2 so as to fasten the body 2 around the limb part. The attachment device 6 is further arranged so that the free end 7 of the extensible part 4 can be attached in a plurality of positions on the body 2 so that the body 2 is more or less tightened onto the limb part so that the body can apply variable pressure on the limb part depending on the attachment position of the free end 7 of the extensible part 4 onto the body 2. Thereby, the tighter the body 2 is around the limb part, the greater the pressure exerted. The attachment device 6 can be of any nature suitable for fulfilling said function. Thus, the attachment device 6 is, e.g., of the self-gripping type with, e.g., at least one area formed of loops or looplets extending on one face of the body 2 and at least one area formed of hooks extending on the opposite face of the body, the loops or looplets cooperating with the hooks when the two faces are applied to each other to ensure attachment. According to one embodiment, the hooks are provided on the inner face of the body 2, i.e., on the face turned towards the limb part of the patient, and the loops are provided on the outer face, i.e., the face turned outward when the compression device is worn. According to a particular embodiment, the entire face of the body 2 comprises loops or looplets while a reduced surface area comprises hooks on the opposite face, e.g., near the end of the extensible part 4. Thus, according to this embodiment, the extensible part 4 can be attached on any point on the body 2, allowing for very fine adjustment of the pressure exerted by the compression device 1.

According to the embodiment shown in FIG. 1, the body 2 has the shape of a band that is entirely extensible and the direction of extensibility of which corresponds to the direction of elongation of the band. In other words, according to this embodiment, the extensible part 4 extends over the entire length of the band. When the compression device is worn, the band surrounds a limb part and is attached on itself by the attachment device 6, as shown in FIG. 1.

According to a particular embodiment, also shown in FIG. 1, the compression device 1 comprises several bodies 2, in the form of parallel bands, attached on each other according to an attachment direction substantially perpendicular to the direction of elongation of the bands. More particularly, the bands are, e.g., sewn together along a seam line extending according to the attachment direction, e.g., substantially in the center of the bands according to the direction of elongation thereof. The bands are, e.g., attached on each other so that one band partially covers the adjacent band, as shown in FIG. 1. The attachment direction corresponds to the direction wherein the limb part on which the compression device 1 is to be applied extends so that each band of the compression device 1 surrounds a specific area of the limb part. The attachment of each band on itself allows for adjusting the pressure exerted on each area of the limb part individually and independently of the pressure exerted on the other areas. In other words, the pressure exerted by the compression device 1 is not necessarily constant over the entire limb part on which this compression device is applied.

According to the embodiment shown in FIG. 2, the compression device 1 comprises a single body 2 comprising an inextensible part 8 and at least one extensible part 4 protruding from an edge 10 of the inextensible part 8. The extensible part 4 extends in projection according to the direction of extensibility thereof and is arranged to be attached on the inextensible part when the body 2 surrounds a limb part, as is more particularly visible in FIG. 3.

According to a particular embodiment, also shown in FIG. 2, the compression device 1 comprises several extensible parts 4 protruding from two opposite edges 10 of the inextensible part 8. The extensible parts 4 then have a paw shape extending on either side of a flank forming the inextensible part 8. According to a direction substantially perpendicular to the direction of extensibility, the extensible parts 4 are alternately arranged on either side of the inextensible part 8. In other words, on the side of the same edge 10 of the inextensible part, there is a space 12 between two extensible parts 4, this space 12 extending at an extensible part 4 protruding from the opposite edge 10 of the inextensible part 8. Thereby, when the compression device is worn, each space 12 is occupied by an extensible part 4 of the other edge 10 of the inextensible part 8, as shown in FIG. 3. The free end 7 of each extensible part 4 can be attached in different positions onto the inextensible part 8 so that the pressure exerted on each area of the limb part can be adjusted individually and independently of the pressure exerted on the other areas. In other words, as for the embodiment of FIG. 1, the pressure exerted by the compression device 1 is not necessarily constant over the entire limb part on which this compression device is applied.

According to the embodiment of FIG. 2, the one or a plurality of extensible parts 4 are made of material with the inextensible part 8, the entire body 2 being made of the same material. Alternatively, different materials may be provided to make the inextensible part 8 and the one or a plurality of extensible parts 4.

Whatever the embodiment, the compression device 1 comprises at least one adjustment device 14 of the pressure exerted by the compression device 1 on the limb part wearing this compression device. The adjustment device 14 indicates at least one level of pressure exerted by the compression device depending on the attachment position of the free end 7 of the extensible part 4, allowing the pressure exerted to be adjusted based on this indication. More particularly, each body 2 of the compression device 1 of the embodiment of FIG. 1 and each extensible part 4 of the compression device 1 of the embodiment of FIG. 2 comprises such an adjustment device 14. In other words, an adjustment device 14 is provided for each area of the limb part for which the pressure exerted by the compression device 1 can be adjusted independently of the other areas.

The adjustment devices 14 are substantially identical, including between the embodiment of FIG. 1 and that of FIG. 2, so that only one adjustment device 14 will now be described.

The adjustment device 14 comprises a tab 16 attached on the body 2 and a loop 18 extending in or on the body 2, of which at least one tab 16 and the loop 18 extends in an extensible part 4.

The tab 16 is made of an inextensible material and extends mainly along the direction of extensibility of the extensible part 4. Along the direction of extensibility, the tab 16 extends between a first end 20 attached on the body 2 and a second end 22 not attached on the body 2. The first end 20 is, e.g., sewn onto the body 2, e.g., on the extensible part 4 near the free end 7 thereof. The second end 22 is, e.g., closer to the free end 7 of the extensible part 4 so that the direction going from the first end 20 to the second end 22 of the tab 16 corresponds to the direction of extensibility of the extensible part 4. In other words, according to one embodiment, the distance separating the first end 20 from the free end 7 of the extensible part 4 is greater than the distance separating the second end 22 from the free end 7 of the extensible part.

The loop 18 extends mainly according to a direction substantially perpendicular to the direction of extensibility. According to an embodiment shown in FIG. 4, the loop 18 is formed by a strip of material 21 attached on the body 2, e.g., by sewing, at the two ends thereof to form an opening extending along the direction of extensibility between the body 2 and the loop 18. According to another embodiment shown in FIG. 5, the loop 18 is formed by two slots 23 made in the body 2, the tab 16 passing through the two slots 23 to pass under the body 2 at the loop 18. According to one embodiment, the loop 18 is attached on or extends in the extensible part 4 of the body 2. Thus, according to one embodiment, the tab 16 and the loop 18 both extend in the extensible part 4, when the body 2 comprises an inextensible part 8. Alternatively, when the body 2 comprises an inextensible part 8, one of the tabs 16 and the loop 18 extends in the extensible part 4 and the other extends in the inextensible part 8.

The tab 16 extends in the loop 18 between the first end 20 thereof and the second end 22 thereof. In other words, the tab 16 passes through the opening formed between the loop 18 and the body 2 between the first end 20 thereof and the second end 22 thereof according to the embodiment of FIG. 4 or under the body 2 at the loop 18 according to the embodiment of FIG. 5. The tab 16 and the loop 18 are movable relative to each other along the direction of extensibility when the extensible part 4 is stretched along the direction of extensibility. In other words, the tab 16 and the loop 18 are in different relative positions in the different attachment positions of the free end 7 of the extensible part 4 on the body 2. More particularly, the distance separating the loop 18 from the second end 22 of the tab 16 varies with the attachment position of the free end 7 of the extensible part 4 on the body 2. Thereby, the position of the tab 16 relative to the loop 18 indicates the level of pressure exerted by the compression device 1 corresponding to the attachment position of the free end 7 of the extensible part 4 onto the body 2. Consequently, by pulling on the extensible part 4 to bring the tab 16 into a particular position relative to the loop 18 and attaching the extensible part 4 on the body 2 when this particular position is reached, a user can adjust the pressure exerted by the compression device 1 on the area of the limb part surrounded by this extensible part 4.

The length of the tab 16, measured along the direction of extensibility from the first end 20 thereof to the second end 22 thereof, is arranged so that the tab 16 remains in the loop 18 regardless of the attachment position of the free end 7 of the extensible part 4 on the body 2. In addition, the width of the loop 18, measured along the direction perpendicular to the direction of extensibility wherein the loop 18 extends, is substantially greater than or equal to that of the tab 16 to ensure the alignment of the tab 16 along the direction of extensibility while allowing the tab 16 to slide in the loop 18 along the direction of extensibility.

According to one embodiment, the second end 22 of the tab 16 comprises a stop element 24 arranged to prevent the extraction of the tab 16 from the loop 18. Thus, in addition to preventing the tab 16 from coming out of the loop 18, the stop element 24 can also serve as a warning to a user when the extensible part 4 has been stretched to the maximum thereof, i.e., the maximum pressure that can be exerted by the body 2 has been reached. The stop element 24 is, e.g., formed by an enlargement of the width of the tab 16 at the second end 22 thereof so that the width of the second end 22 is greater than the width of the loop 18, as more particularly visible in FIGS. 4 and 5.

According to an embodiment shown in FIGS. 4 and 5, the tab 16 comprises at least one index 26 placed between the first end 20 and the second end 22. The position of the index 26 relative to the loop 18 indicates the level of pressure exerted by the compression device 1 corresponding to the attachment position of the free end 7 of the extensible part 4 onto the body 2. In other words, the index 26 can serve as a marker to determine the pressure exerted by the compression device 1 based on the distance separating the index 26 from the loop 18 when the free end 7 of the extensible part 4 is attached on the body 2. According to one embodiment, the index 26 indicates a predetermined level of pressure exerted by the compression device 1 when the free end 7 of the extensible part 4 is attached on the body 2 while the index 26 is aligned with the loop 18. The index 26 is, e.g., formed by a visual and/or tactile mark extending according to the width of the tab 16, such as a line, e.g., of a different color from that of the tab 16 and/or extending in relief or recessed from the tab 16, as shown in FIGS. 4 and 5. It is understood that other forms of index can be envisaged, such as a graduated scale extending along the tab 16 and indicating different pressure values or other.

According to a particular embodiment, the tab 16 comprises at least two indices 26, spaced apart along the direction of extensibility of the extensible part 4, the position of each index 26 corresponding to a predetermined level of pressure exerted by the compression device 1 when the index 26 is aligned with the loop 18. In other words, each index 26 corresponds to an attachment position of the free end 7 of the extensible part 4 onto the body 2 wherein the compression device 1 applies a predetermined pressure on the limb part. As shown in FIGS. 4 and 5, the tab 16 can comprise more than two indices 26 to form a graduation indicating the pressure exerted by the compression device according to the index 26 aligned with the loop 18.

According to one embodiment, the different indices can be of different colors and/or textures to provide additional information to the user. Thus, the colors and/or textures can be representative of the pressure exerted by the compression device according to the index 26 aligned with the loop 18. For example, the indices 26 can be increasingly darker as the corresponding pressure is higher. Alternatively, a color code can be adopted, with green representing, e.g., the lowest pressure and red the highest pressure.

Each index represents, e.g., a range of pressures exerted by the compression device according to the index aligned with the loop 18. Thus, according to a particular embodiment, the tab 16 comprises three indices representing successively a pressure range between 20 mmHg and 30 mmHg, between 30 mmHg and 40 mmHg, and between 40 mmHg and 50 mmHg. These indices are, e.g., of different colors and/or textures, as indicated previously. It is understood that indices representing other ranges and/or additional ranges may be provided.

Thereby, by using the adjustment device 14, a user can adjust in a simple and particularly accessible way, the pressure exerted by at least a part of the compression device 1. Indeed, when the user pulls on the extensible part 4, this causes a relative movement of the tab 16 relative to the loop 18 along the direction of extensibility, the tab being inextensible. When the tab 16 is in the desired position relative to the loop 18, this position being, e.g., marked by an index 26, the user can attach the free end 7 of the extensible part 4 onto the body 2 to apply on the limb part, the pressure corresponding to this position of the tab 16 relative to the loop 18.

As previously indicated, and as illustrated in FIG. 3, the user can independently adjust the pressure exerted on different areas of the limb part by means of several adjustment devices 14 corresponding to each body 2 or to each extensible part 4 of the compression device. Depending on the desired pressure profile, all adjustment devices 14 may be set to be in the same position, which does not necessarily correspond to a uniform pressure exerted by the compression device 1 because this pressure also depends on the diameter of the limb part, or to be in different positions to exert more or less significant pressures depending on the area of the limb part concerned.

The one or a plurality of adjustment devices 14 may be used during the application of the compression device 1, but also to tighten the compression device if it is found that a tab 16 has moved relative to a loop 18, e.g., after a certain duration of application of the compression device 1. Indeed, the adjustment device 14 allows for easily reproducible pressure adjustment.

The adjustment device 14 described above is particularly easy to use and accessible for the person applying the compression device, including when this person is the patient themselves. In addition, the pressure adjustment is easily reproducible, as previously described.

1. A therapeutic compression device for a part of the limb of a patient, comprising at least one body at least partially extensible, a free end of an extensible part of the body being attachable in a plurality of attachment positions on the body around the limb part, the pressure exerted by the compression device varying according to the position of the free end of the extensible part on the body, said compression device further comprising at least one adjustment device of the pressure exerted by the compression device indicating at least one level of pressure exerted by the compression device according to the attachment position of the free end of the extensible part, wherein said adjustment device comprises at least one inextensible tab attached on the body and at least one loop extending on or in the body, at least one of the tab and the loop extending in the extensible part, said tab being engaged in said loop and moving relative to the loop along the direction of extensibility of the extensible part depending on the attachment position of the free end of the extensible part, the position of the tab relative to the loop indicating the level of pressure exerted by the compression device corresponding to the attachment position of the free end of the extensible part on the body.

2. The compression device according to claim 1, wherein the tab and the loop extend in the extensible part of the body.

3. The compression device according to claim 1, wherein the tab comprises at least one index, the position of the index relative to the loop indicating the level of pressure exerted by the compression device corresponding to the attachment position of the free end of the extensible part on the body.

4. The compression device according to claim 3, wherein the tab comprises at least two indices, spaced apart along the direction of extensibility of the extensible part, the position of each index corresponding to a predetermined level of pressure exerted by the compression device when said index is aligned with the loop.

5. The compression device according to claim 1, wherein the tab extends between a first end attached on the body and a second end not attached on the body, the tab extending in the loop between its first end and its second end.

6. The compression device according to claim 5, wherein the second end of the tab comprises a stop element preventing the extraction of the tab from the loop.

7. The compression device according to claim 1, wherein the body is formed by an extensible band that can be wrapped around the limb part and attached on itself in a plurality of attachment positions around the limb part.

8. The compression device according to claim 7, comprising several bodies each formed by an extensible band, said bodies being attached on each other and each being able to be wrapped around a different limb part, each body comprising a pressure adjustment device.

9. The compression device according to claim 1, wherein the body comprises an inextensible part and at least one extensible part, the extensible part protruding from an edge of said inextensible part and being attachable in a plurality of attachment positions on the inextensible part.

10. The compression device according to claim 9, wherein the body comprises a plurality of extensible parts protruding from at least one edge of the inextensible part, each extensible part being attachable in a plurality of attachment positions on the inextensible part, a pressure adjustment device being provided for each extensible part.
